(19) United States
(12) Patent Application Publication
(10) Pub. No.: US 2025/0049616 A1
(43) Pub. Date: Feb. 13, 2025

(54) Diaper Guard Device

(71) Applicant: Nathanael Paul Jenkins, Anchorage, AK (US)

(72) Inventor: Nathanael Paul Jenkins, Anchorage, AK (US)

(21) Appl. No.: 18/665,719

(22) Filed: May 16, 2024

Related U.S. Application Data
(60) Provisional application No. 63/532,048, filed on Aug. 10, 2023.

(51) Int. Cl. A61F 13/49; A61F 13/15; A61F 13/58

(52) U.S. Cl. CPC A61F 13/49058; A61F 13/15203; A61F 13/58

(57) ABSTRACT

A diaper guard device is provided. The device is designed for placement on a user's body beneath a diaper to shield the genital area from contact with waste, thereby preventing rashes and infections. The device comprises a body made from a medical-grade, absorbent, and anti-bacterial fabric material, wherein the body generally features a first end with one or two tabs and a second end with at least one tab for secure attachment. These tabs, along with a skin-safe, water-resistant, or waterproof adhesive fastener located on the rear surface of the body, facilitate the attachment of the device to the user's skin, ensuring that it remains in place to provide the intended protective barrier. During use, the device does not inhibit waste movement within the diaper but effectively prevents direct contact of waste with the genital area, thereby reducing the risk of skin irritation and infections.

### CROSS-REFERENCE TO RELATED APPLICATION

The present application claims priority to, and the benefit of, U.S. Provisional Application No. 63/532,048, which was filed on Aug. 10, 2023, and is incorporated herein by reference in its entirety.

### FIELD OF THE INVENTION

The present invention relates generally to the field of diapers. More specifically, the present invention relates to a diaper guard device that can be adhered around the genital area of a user underneath a diaper to prevent waste from within the diaper from causing rashes and/or infections. Accordingly, the present disclosure makes specific reference thereto. Nonetheless, it is to be appreciated that aspects of the present invention are also equally applicable to other like applications, devices, and methods of manufacture.

### BACKGROUND

When feces and urine are not effectively contained within a diaper, they can remain in prolonged contact with the wearer's skin, particularly in the genital and buttock areas. This contact can lead to several health issues. Firstly, urine contains urea, which breaks down into ammonia, elevating the skin's pH and weakening its natural protective barrier, thereby increasing susceptibility to irritation and infection. Additionally, both urine and feces create a moist environment that compromises skin integrity and promotes the growth of bacteria and fungi, potentially leading to infections.

Feces is the most problematic as they contain enzymes that can irritate and break down the skin's protective layers, leading to diaper rash. Movement exacerbates the issue by causing friction between the diaper and the skin, especially when the diaper is wet or full. This friction, combined with the irritants in feces, can lead to chafing, further irritating the skin.

Moreover, the warm, moist environment of a soiled diaper is an ideal breeding ground for bacteria and fungi. These microorganisms can cause infections such as diaper dermatitis and yeast infections, which are not only uncomfortable but can also be challenging to treat.

Therefore, there exists a long-felt need in the art for a device that provides improved diaper hygiene. There also exists a long-felt need in the art for a diaper guard device. More specifically, there exists a long-felt need in the art for a diaper guard device that prevents feces from contacting the genital area of a wearer within a diaper.

The subject matter disclosed and claimed herein, in one embodiment thereof, comprises a diaper guard device. The device is designed for placement on a user's body beneath a diaper to shield the genital area from contact with waste, thereby preventing rashes and infections. The device comprises a body made from a medical-grade, absorbent, and anti-bacterial fabric material, wherein the body generally features a first end with one or two tabs and a second end with at least one tab for secure attachment. These tabs, along with a skin-safe, water-resistant, or waterproof adhesive fastener located on the rear surface of the body, facilitate the attachment of the device to the user's skin, ensuring that it remains in place to provide the intended protective barrier. During use, the device does not inhibit waste movement within the diaper but effectively prevents direct contact of waste with the genital area, thereby reducing the risk of skin irritation and infections.

In this manner, the diaper guard device of the present invention accomplishes all the foregoing objectives and provides a device that provides improved diaper hygiene. More specifically, the device prevents feces from contacting the genital area of a wearer within a diaper. This reduces the risk of diaper rash and/or skin infection.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a diaper guard device that can be attached to a user's body underneath a diaper. More specifically, the body of the device covers the user's genital area such that waste within the diaper does not contact said genital area.

The body of the device is preferably made from a medical-grade fabric material that is absorbent. Said material is preferably anti-bacterial to prevent rashes and infections. The body is preferably comprised of a first end with at least one (but preferably two) tab(s) and a second end with at least one tab. The tabs provide attachment areas for the device to be attached to the genital area of the user.

More specifically, the body attaches to the genital area of the user via at least one fastener located on the rear surface of the body. The fastener is preferably a skin-safe adhesive that is water-resistant and/or waterproof. As a result, the fastener allows for attachment of the body and tabs to the user's skin. After adhesion to a user's skin, the device prevents rashes and infections from forming on the user's genital area due to contact with waste within a diaper.

The device is also comprised of a method of using the device. First, a device is provided comprised of a body with a rear surface comprised of at least one fastener. Then, the body is secured to a user's skin around a user's genital area via the fastener such that the device provides a seal around the genital area. Then, a diaper can be placed over the device.

Accordingly, the diaper guard device of the present invention is particularly advantageous as it provides a device that provides improved diaper hygiene. More specifically, the device prevents feces from contacting the genital area of a wearer within a diaper. This reduces the risk of diaper rash and/or skin infection. In this manner, the diaper guard device overcomes the limitations of existing diapers known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one potential embodiment of a diaper guard device of the present invention in accordance with the disclosed architecture;

FIG. 2 illustrates a front perspective view of one potential embodiment of a diaper guard device of the present invention in accordance with the disclosed architecture;

FIG. 3 illustrates a rear perspective view of one potential embodiment of a diaper guard device of the present invention in accordance with the disclosed architecture; and

FIG. 4 illustrates a flowchart of a method of using one potential embodiment of a diaper guard device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a device that provides improved diaper hygiene. There also exists a long-felt need in the art for a diaper guard device. More specifically, there exists a long-felt need in the art for a diaper guard device that prevents feces from contacting the genital area of a wearer within a diaper.

The present invention, in one exemplary embodiment, is comprised of a diaper guard device. The device can be attached to a user's body underneath a diaper such that it covers the user's genital area such that waste within the diaper does not contact said genital area.

The body of the device is preferably made from a medical-grade fabric material that is absorbent and anti-bacterial to prevent rashes and infections. The body is preferably comprised of a first end with at least one (but preferably two) tab(s) and a second end with at least one tab. The tabs provide attachment areas for the device to be attached to the genital area of the user.

More specifically, the body attaches to the genital area of the user via at least one fastener located on the rear surface of the body. The fastener is preferably a skin-safe adhesive that is water-resistant and/or waterproof. As a result, the fastener allows for attachment of the body and tabs to the user's skin. After adhesion to a user's skin, the device prevents rashes and infections from forming on the user's genital area due to contact with waste within a diaper.

The device is also comprised of a method of using the device. First, a device is provided comprised of a body with a rear surface comprised of at least one fastener. Then, the body is secured to a user's skin around a user's genital area via the fastener such that the device provides a seal around the genital area. Then, a diaper can be placed over the device.

Accordingly, the diaper guard device of the present invention is particularly advantageous as it provides a device that provides improved diaper hygiene. More specifically, the device prevents feces from contacting the genital area of a wearer within a diaper. This reduces the risk of diaper rash and/or skin infection. In this manner, the diaper guard device overcomes the limitations of existing diapers known in the art.

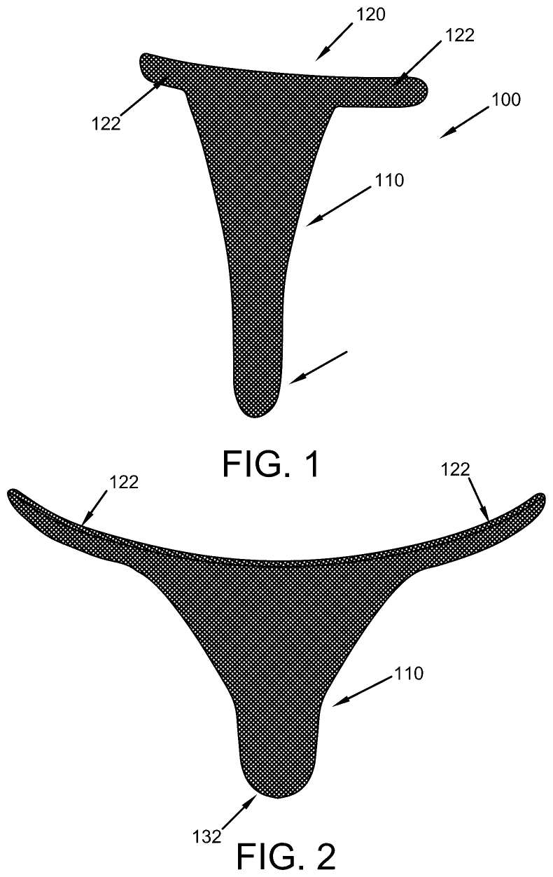

Referring initially to the drawings, FIG. 1 illustrates a front perspective view of one potential embodiment of a diaper guard device 100 of the present invention in accordance with the disclosed architecture. During use, the device 100 is attached to a user's body underneath a diaper. The body 110 of the device 100 covers the user's genital area such that waste within the diaper does not contact said genital area. This prevents rashes and infections.

The body 110 of the device 100 is preferably made from a medical-grade fabric material that is absorbent such that the device 100 absorbs urine and filters said urine, through the anti-bacterial material that makes up the body 110, to prevent rashes and infections. The body 110 may be any shape in different embodiments. However, the body 110 is preferably comprised of a first end 120 with at least one (but preferably two) tab(s) 122 and a second end 130 with at least one tab 132, as seen in FIG. 2. The tabs 122, 134 provide attachment areas for the device 100 to be attached to the genital area of the user.

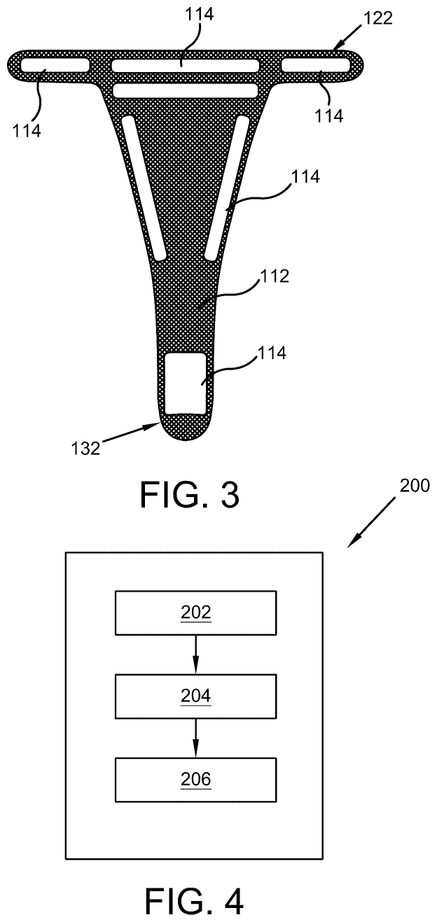

More specifically, the body 110 attaches to the genital area of the user via at least one fastener 114 located on the rear surface 112 of the body 110, as seen in FIG. 3. The fastener 114 is preferably a skin-safe adhesive that is water-resistant and/or waterproof. As a result, the fastener 114 allows for attachment of the body 110 and tabs 122, 132 to the user's skin. The fastener 114 may be positioned anywhere on the rear surface 112, including on the tabs 122, 132.

After adhesion to a user's skin, the device 100 prevents rashes and infections from forming on the user's genital area due to contact with waste within a diaper. It should be noted that the device 100 does not stop waste from traveling within a diaper and only stops waste from contacting the user's genital area.

The device 100 is also comprised of a method of using 200 the device 100, as seen in FIG. 4. First, a device 100 is provided comprised of a body 110 with a rear surface comprised of at least one fastener 114 [Step 202]. Then, the body 110 is secured to a user's skin around a user's genital area via the fastener 114 such that the device 100 provides a seal around the genital area [Step 204]. Then, a diaper can be placed over the device 100 and around the user [Step 206].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “diaper guard device” and “device” are interchangeable and refer to the diaper guard device 100 of the present invention.

Notwithstanding the foregoing, the diaper guard device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the diaper guard device 100 as shown in the FIGS. are for illustrative purposes only, and that many other sizes and shapes of the diaper guard device 100 are well within the scope of the present disclosure. Although the dimensions of the diaper guard device 100 are important design parameters for user convenience, the diaper guard device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A diaper guard device comprising:
a body comprised of a rear surface comprised of a fastener.

2. The diaper guard device of claim 1, wherein the body is comprised of a fabric material.

3. The diaper guard device of claim 2, wherein the fabric material is comprised of an anti-bacterial material.

4. The diaper guard device of claim 2, wherein the fabric material is comprised of an absorbent material.

5. The diaper guard device of claim 1, wherein the fastener is comprised of an adhesive fastener.

6. The diaper guard device of claim 5, wherein the fastener is comprised of a water-resistant fastener.

7. The diaper guard device of claim 5, wherein the fastener is comprised of a waterproof fastener.

8. The diaper guard device of claim 5, wherein the fastener is comprised of a skin-safe adhesive.

9. A diaper guard device comprising:
a body comprised of:
a first end comprised of a first tab;
a second end comprised of a second tab; and
a rear surface comprised of a fastener.

10. The diaper guard device of claim 9, wherein the body is comprised of a fabric material.

11. The diaper guard device of claim 10, wherein the fabric material is comprised of an anti-bacterial material.

12. The diaper guard device of claim 10, wherein the fabric material is comprised of an absorbent material.

13. The diaper guard device of claim 9, wherein the fastener is comprised of an adhesive fastener.

14. The diaper guard device of claim 13, wherein the fastener is comprised of a water-resistant fastener.

15. The diaper guard device of claim 13, wherein the fastener is comprised of a waterproof fastener.

16. The diaper guard device of claim 13, wherein the fastener is comprised of a skin-safe adhesive.

17. The diaper guard device of claim 9, wherein the rear surface is the rear surface of the first tab.

18. The diaper guard device of claim 9, wherein the rear surface is the rear surface of the second tab.

19. A method of using a diaper guard device, the method comprising the following steps:
providing a diaper guard device comprised of a body comprised of a rear surface comprised of a fastener;
securing the body to a user's skin around the user's genital area via the fastener; and
placing a diaper over the diaper guard device around the user.

20. The method of using a diaper guard device of claim 19, wherein the body is comprised of an anti-bacterial, absorbent, medical grade fabric.
